(19) United States
(12) Patent Application Publication
(10) Pub. No.: US 2010/0273205 A1
(43) Pub. Date: Oct. 28, 2010

(54) Magnetic resonance imaging contrast agents

(76) Inventors: Megan L. Blackwell, Cambridge, MA (US); Bruce R. Rosen, Lexington, MA (US); Christian T. Farrar, Arlington, MA (US); Wellington Pham, Brentwood, TN (US)

Correspondence Address: QUARLES & BRADY LLP, 411 E. WISCONSIN AVENUE, SUITE 2040, MILWAUKEE, WI, 53202-4497, US

(21) Appl. No.: 12/803,616

(22) Filed: Jun. 30, 2010

Related U.S. Application Data
(62) Division of application No. 11/430,701, filed on May 8, 2006.
(60) Provisional application No. 60/678,356, filed on May 6, 2005.

(51) Int. Cl. C12Q 1/02; C09B 21/00; C09B 47/04

(52) U.S. Cl. 435/29; 534/16; 540/140

(57) ABSTRACT

This invention relates to novel magnetic resonance imaging contrast agents, and methods of making and use thereof.

### CLAIM OF PRIORITY

This application is a divisional application of U.S. patent application Ser. No. 11/430,701 filed May 8, 2006, which claims the benefit under 35 U.S.C. §119(e) of the filing date of U.S. Provisional Application No. 60/678,356, filed on May 6, 2005, the entire contents which are incorporated by reference herein.

### FEDERALLY SPONSORED RESEARCH OR DEVELOPMENT

This invention was made with Government support under Grant No. NIBIB 5T32EB001680-03 awarded by the National Institutes of Health. The Government has certain rights in the invention.

### TECHNICAL FIELD

This invention relates to novel magnetic resonance imaging contrast agents, and methods of use thereof.

### BACKGROUND

Higher magnetic field strengths and more powerful gradient systems developed in recent years have resulted in increased signal-to-noise ratios (SNR) in magnetic resonance (MR) images. The increased SNR may be used to acquire images of high resolution, even including isotropic voxels around 1 mm. This resolution may be increased to about 100 microns for ex vivo samples because of even higher SNR achievable from long scan sessions and receiver coil placement directly on the sample. Ex vivo imaging is a powerful tool for pathologists, permitting non-destructive analysis of tissues, and for neuroimagers who may use probabilistic maps of brain regions created from ex vivo data sets to inform their in vivo scans. Further, ex vivo imaging serves as a bridge between in vivo imaging and histology, illuminating the cytoarchitectonic and myeloarchitectonic contributions to the MR signal.

### SUMMARY

The invention is based, in part, on the discovery that the use of magneto-optical contrast agents that include a tissue-specific histological dye and a paramagnetic core can enhance contrast in ex vivo magnetic resonance imaging of tissues.

Thus, in one aspect, the invention provides magneto-optical contrast agent compositions that include a paramagnetic core chemically linked via a chelating moiety to a tissue-specific histological dye.

In some embodiments, the tissue-specific histological dye is selected from the group consisting of cresyl violet, toluidine blue, neutral red, thionine, and chromoxane cyanine R. The dye can be, e.g., a neural tissue-specific dye. For example, a neural tissue-specific dye can preferentially stain one or more of white matter, gray matter, neurons, neuronal cell bodies, axons, glia, or myelin. A dye that “preferentially” stains is one that stains detectably more in certain areas, e.g., areas that contain white matter, gray matter, neurons, neuronal cell bodies, axons, glia, or myelin, and does not exhibit substantial non-specific staining. In some embodiments, the tissue-specific histological dye is thionine.

In some embodiments, a linker can be used between the chelating moiety and the histological dye. The linker can include, e.g., one or more carbon atoms, e.g., 2 to 7 carbon atoms.

In some embodiments, the paramagnetic core is a rare earth metal, e.g., a lanthanide. In some embodiments, the paramagnetic core is selected from the group consisting of copper, gadolinium, manganese, and iron.

In some embodiments, the chelating moiety is a linear or macrocyclic chelating moiety, e.g., 1,4,7,10-tetraazacyclododecane-N,N′,N″,N′″-tetraacetic acid (DOTA), ethylenediamine tetraacetic acid (EDTA), or diethylene triamine pentaacetate (DTPA).

In some embodiments, the composition is or includes C₂₈H₃₃GdN₇O₇S.

In another aspect, the invention provides methods for making a magneto-optical contrast agent as described herein. The methods include chemically linking a paramagnetic core to a tissue-specific histological dye via a chelating moiety, thereby forming the contrast agent. In some embodiments, the methods include linking the chelating moiety to the histological dye via a linker comprising one or more carbon atoms.

In a further aspect, the invention provides methods for obtaining an ex vivo magnetic resonance image of a specific tissue. The methods include contacting a sample including the tissue (e.g., a tissue slice or biopsy) with a magneto-optical contrast agent as described herein, e.g., a magneto-optical contrast agent including a paramagnetic core and a tissue-specific histological dye, e.g., under conditions and for a time sufficient for the contrast agent to substantially penetrate the tissue, and imaging the sample using magnetic resonance, thereby obtaining an image of the tissue.

In some embodiments, the contrast agent is or includes luxol fast blue.

In some embodiments, the contrast agent includes a paramagnetic core chemically linked via a chelating moiety to a tissue-specific histological dye.

In some embodiments, the contrast agent includes gadolinium and thionine.

In some embodiments, the contrast agent selectively reduces relaxation parameters of the sample when compared to a control sample, e.g., reduces spin-lattice relaxation time (T1), and/or spin-spin relaxation time (T2).

In some embodiments, the contrast agent selectively increases signal-to-noise ratio of the imaged sample when compared to a control sample imaged in the absence of the contrast agent.

In some embodiments, the methods further include performing a histological examination of the sample.

As used herein, the term “histological stain” or “histological dye” refers to a chemical compound that, when contacted with a tissue, imparts a detectable label to the tissue. A “tissue-specific” histological dye is one that preferentially labels a particular tissue (e.g., a particular cell structure, or cell type). The dyes useful in the methods and compositions described herein are generally optically detectable, e.g., using fluorescent or standard light optics. Some of the dyes may be detectable on their own, while others will require further processing, e.g., enzymatic or other processing, such as treatment by a differentiating solution that will remove any unbound dye. The terms “stain” and “dye” are used interchangeably herein.

The compositions and methods described herein provide for tissue-specific contrast enhancement in ex vivo magnetic resonance imaging. The disclosed compositions selectively enhance relaxation parameters of tissues, thereby increasing contrast and allowing finer resolution of structures, e.g., in complex tissues such as the brain.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1C are a series of bar graphs showing relaxation parameters of diethylenetriaminepentaacetate (DTPA⁵⁻) chelate of gadolinium(III) [Gd-DTPA]. FIG. 1A is a graph of changes in T1 in gray matter (black bars) and white matter (white bars) as a function of gadolinium concentrations at 7T. FIG. 1B is a graph of changes in T2 as a function of gadolinium concentrations. FIG. 1C is a graph of contrast-to-noise ratios (CNR, the ratio of the difference of mean signals in the two regions of interest to the standard deviation of the background signal).

FIG. 2 is a chemical structure of Luxol fast blue (LFB), where X is SO3⁻

FIGS. 3A-3C are a series of inversion-recovery prepared spin echo images of ex vivo visual cortex. The images were acquired at 14T. The inversion times chosen for contrast were: 305 ms, 105 ms and 720 ms. FIG. 3A is a control image of formalin-fixed visual cortex. FIG. 3B is an image of ethanol-immersed and lithium-carbonate-treated visual cortex. FIG. 3C is an image of luxol-fast-blue-immersed and lithium-carbonate-treated visual cortex.

FIGS. 3D-3E are a pair of bar graphs showing the changes in T1 (3D) and T2 (3E) with LFB and ethanol control, in outer gray matter (black bars), inner gray matter (gray bars), and white matter (white bars).

FIGS. 4A and 4B are a series of graphs showing longitudinal relaxation times for white matter (WM) and gray matter (GM) in ex vivo visual cortex with various preparations. Control samples have only been formalin-fixed; eth refers to 70% ethanol solution; Li₂CO₃ refers to lithium carbonate and LFB refers to luxol fast blue. FIG. 4A is a graph showing IR-SE signal intensity differences between control and luxol-fast-blue-treated samples. FIG. 4B is a graph showing IR-SE signal intensity differences between control and ethanol-immersed and lithium-carbonate-treated samples.

FIGS. 5A-D are a series of photomicrographs of human visual cortex stained with LFB, at different magnifications. 5A is a 2× image. 5B, a 10× image of the area in the yellow box on 5A. 5C is a 40× image of the area in the green box on 5A, and 5D is a 40× image of the area in the white box on 5A.

FIG. 6A is a chemical structure of thionine, a Nissl dye used to visualize cells such as neurons.

FIG. 6B is a schematic illustration of a procedure for synthesizing gadolinium-thionine.

FIG. 7A is a graph of measured relaxivity of gadolinium-thionine. FIG. 7B is a 2× microscope image of rat hippocampus stained with gadolinium-thionine. FIG. 7C is a digital image of rat hippocampus stained with regular thionine.

FIG. 8 is a schematic illustration of a procedure for synthesizing gadolinium-thionine including a linker between the dye and the DOTA.

FIG. 9 is a schematic illustration of a procedure for synthesizing optical-magneto contrast agents that include cresyl violet, toluidine blue, or neutral red linked to Gd-conjugated DOTA.

FIGS. 10 and 11 are bar graphs illustrating the change in CNR in tissues stained with formalin, LFB, GD-DTPA, and MnCl for T1 (FIG. 10) and T2* (FIG. 11).

### DETAILED DESCRIPTION

Histology methods allow study of the cytoarchitecture of the complex tissues, such as the human brain. Ex vivo magnetic resonance imaging is also a promising tool for analyzing tissue samples. Technology combining these two methods, as described herein, provides a tool to assist neuroscientists in understanding the substructure and function of complex tissues such as the brain, and to assist pathologists in uncovering structural abnormalities, e.g., abnormalities in brain structure.

### Magneto-Optical Contrast Agents

The methods described herein improve contrast and SNR using extrinsic magneto-optical contrast agents to enhance the relaxation times of specific regions of interest. Non-specific contrast agents have been used for ex vivo imaging, including gadolinium-based agents (Johnson et al., Radiology, 222:789-793 (2002)); A. F. Mellin et al., Mag. Reson. Med., 32(2):199-205 (1994)); Smith et al., Proc. Natl. Acad. Sci. USA, 91(9):3530-3533 (1994)); Jacobs and Fraser, J. Neur. Met., 54(2):189-196 (1994)). Johnson et al. (Johnson et al., Radiology, 222:789-793 (2002))) have achieved six-fold improvement in SNR using diethylenetriaminepentaacetate (DTPA⁵⁻) chelate of gadolinium(III) [Gd-DTPA], but the biological specificity of the agents was unsatisfactory. Johnson et al., Radiology, 222: p. 789-793 (2002), showed that staining many tissues with a 20:1 mixture of formalin and gadopentate dimeglumine reduced the T1 values from their initial value, which ranged between 800 and 2000 ms at a field strength of 2 Tesla, to approximately 100 ms. Johnson states, “At this point, MR contrast enhancement is empirical. The relationship to specific biologic structure is not clear.” Manganese chloride (MnCl) has been used in living animals, but also lacks tissue specificity (Pautler et al., Magn. Reson. Med. 70:740-748 (1998)). Lowe, Current Pharmaceutical Biotechnology, 5:519-528 (2004), noted the need to move away from the “nonspecificity of these earlier contrast agents.” The magneto-optiocal contrast agents described herein have a more specific distribution than the existing gadolinium chelates.

MR contrast agents generally affect the relaxation times, longitudinal T1 and transverse T2, of the tissue of interest. SNR is increased by T1-shortening agents that permit more scans to be performed and averaged in the same amount of time. SNR is defined as the ratio of the mean signal in a given region of interest to the standard deviation of the background signal. CNR is defined as the ratio of the difference of mean signals in two regions of interest to the standard deviation of the background signal. It can be seen that maximal SNR for a tissue class does not necessarily translate to maximal CNR between the given tissue class and another tissue class. An ideal contrast agent would selectively reduce the T1 or T2 of a specific tissue class or have a much greater effect on one tissue class than others. The magneto-optical contrast agents disclosed herein show desirable tissue specificity.

The magneto-optical contrast agents described herein include tissue-specific histological dyes. A number of tissue-specific histological dyes, and methods for using them, are detailed in Kiernan, Histological and Histochemical Methods: Theory and Practice (Pergamon Press, 1990). The magneto-optical contrast agents described herein also include paramagnetic cores, either linked to the dye via a chelating moiety, or intrinsic to the dye. LFB is an example of a magneto-optical contrast agents that includes a paramagnetic core, e.g., copper.

Tissue-Specific Histological Dyes

Histological dyes that are useful in the magneto-optical contrast agents described herein are those that are tissue specific and either (i) include a functional group, e.g., a reactive amino group, that is available for bioconjugation of a paramagnetic core, or (ii) include a paramagnetic core as part of the dye.

Dyes in the first category include cresyl violet; toluidine blue, neutral red, thionine, and chromoxane cyanine R. These dyes possess reactive amino groups that can be conjugated to another amino group on a linker molecule, which will react with a chelating moiety. A linker can be used, e.g., when a dye's low nucleophilicity hinders direct conjugation with a chelate, or if the chelate affects the tissue-specificity of the dye.

As noted above, LFB is an example of the second kind of dye, as it includes a porphyrin group that chelates a metal ion, e.g., a copper ion. LFB can be modified to include other paramagnetic cores as well, e.g., iron(II), manganese(II), or manganese(III).

Paramagnetic Cores

The contrast agents described herein include paramagnetic cores, e.g., one or more paramagnetic metal ions. Paramagnetism results from the presence of unpaired electrons, and is a physical property of substantial magnitude that can be measured using magnetic susceptibility measurements. The greater the number of unpaired electrons, the larger the paramagnetic moment.

The most widely used contrast agents for magnetic resonance imaging are forms of gadolinium, followed by iron oxide and manganese chloride. These paramagnetic cores, as well as the rare earth elements listed below, can be used in the compositions described herein. Rare earth compounds have atomic numbers between 57 and 71 and are usually highly paramagnetic. Furthermore, the lanthanide series, a subset of rare earth elements, have coordination numbers greater than six and sometimes as high as 12.

The coordination number is the number of donor atoms that surround a metal ion. The rare earth elements include Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), and Lutetium (Lu).

Chelating Moieties

In some embodiments, the contrast agents described herein include chelating moieties. Chelates are separate moieties that link a paramagnetic core to a tissue-specific histological dye. Suitable chelates include linear and macrocyclic chelating moieties, e.g., 1,4,7,10-tetraazacyclododecane-N,N′,N″,N′″-tetraacetic acid (DOTA), ethylenediamine tetraacetic acid (EDTA), and diethylene triamine pentaacetate (DTPA); other suitable macrocyclic chelating moieties are known in the art, e.g., as described in Mishra and Chatal, New J. Chem., 25:336-339 (2001), and Takenouchi et al., J. Org. Chem. 58:6895-6899 (1993).

Methods of Making the Magneto-Optical Contrast Agents

Standard synthetic chemical methods can be used to make the magneto-optical contrast agents described herein. For example, the dye can be first attached to a chelate and then the paramagnetic core is added. An exemplary process is described below in Examples 5-7. For example, 4,7,10-Tris-tert-butoxycarbonylmethyl-1,4,7,10-tetraaza-cyclododec-1-yl)-acetic acid (DOTA) can be treated with excess dicyclohexylcarbodiimide (DCC) and N-hydroxysuccinimide (NHS) in dimethylformamide (DMF) in the presence of diisopropylethyl amine (DIEA) followed by the addition of the dye. After Boc group deprotection in TFA, a suitable compound including the paramagnetic core, e.g., gadolinium chloride hexahydrate, is added. Mass spectroscopy can be used to confirm the product.

In some embodiments, additional linkers, e.g., a coupling agent such as N-hydroxysuccinimide (NHS), can be supplied for coupling the chelator, e.g., DOTA, to the dye. In some embodiments, linkers comprising one or more, e.g., 6 to 12, carbon atoms, are included between the chelating moiety and the histological dye. See, e.g., Examples 6-7.

### Ex Vivo MR Imaging—General Methodology

High-resolution ex vivo magnetic resonance (MR) imaging holds great potential for measuring histological properties of any tissue. In many ways, MR is a superior alternative to histology. It is non-destructive, and acquisition of three-dimensional data sets permit “reslicing” the sample in any desired orientation. In most histological preparations, a tissue sample may be stained for one feature and counterstained for only one or two more features. Additional histological measurements cannot be performed on the same sample, as the chemical treatments are irreversible. MR does not suffer from these setbacks, as MR methods do not alter the tissue sample, and MR imaging permits repeated measurements with numerous forms of intrinsic and potential extrinsic contrasts based on proton density and relaxation times. As imaging technology improves and permits smaller voxel sizes, a potential limitation of this ultra-high resolution imaging is poor SNR. SNR is proportional to voxel volume, so as the standard in vivo voxel size of 1×1×5 mm³, a volume equivalent of 5 μL is diminished to an isotropic voxel size of 100 μm, a volume equivalent of 10⁻³ μL, SNR is decreased by a factor of 10³. This loss can be recovered by using the magneto-optical contrast agents described herein.

In general, in the methods described herein, tissue samples are immersed in a solution including a magneto-optical contrast agent as described herein, and imaged using standard MR imaging methods. The imaging can be repeated more than once, e.g., at daily intervals until the relaxation values have reached equilibrium. Relaxation rates can be estimated using inversion-recovery spin-echo and multi-echo spin-echo sequences and nonlinear least-squares fitting routines, as previously described.

Concentrations of magneto-optical contrast agents used in the methods described herein can be determined experimentally, e.g., by using increasing concentrations of the agent until either a saturation limit or SNR limit, caused by excessive shortening of T2, is reached. The same samples can be immersed in increasing agent concentrations to aid image region of interest (ROI) comparison.

In some embodiments, before imaging each specimen that has been incubated in a magneto-optical contrast agent, the sample can be blotted to remove excess agent and then washed, e.g., with phosphate buffered saline. Next, the samples can be blotted and then immersed in perfluoropolyether in a low-pressure environment. The perfluoropolyether is used as an embedding material because it does not contain hydrogen atoms that would contribute to the MR signal. The low-pressure environment is used to remove artifact-inducing air bubbles from within the tissue.

Relaxivity calculations are generally performed by fitting a line to the pre- and post-contrast relaxation values as a function of magneto-optical contrast agent concentration. Once the longitudinal and transverse relaxivities of the contrast agent have been determined, a contrast agent concentration is selected that sufficiently shortens the T1 of the region of interest compared to its surroundings without undesirably shortening T2.

Next, tissue samples are immersed in this optimal concentration of magneto-optical contrast agent until the agent has sufficiently penetrated tissue. A differentiating solution is then applied to the stained tissue, if required. Stained, differentiated tissues are then scanned at high resolutions, typically 40-100 microns isotropic volume,

Optimization routines are conducted across the range of contrast agent concentrations used, and the scan parameters that yield the greatest sum of squared contrast-to-noise ratio (CNR) between structures are determined. This CNR figure-of-merit is compared among all agent concentrations and the concentration producing the largest CNR is considered optimal.

Should any tissue structure not be distinguished from neighboring structures, the sequence parameters can be reoptimized to maximize CNR, e.g., between the structure and any indistinguishable borders. Images can then be reacquired with new parameters and the resolution analysis repeated. The cell types in both MR-visible and MR-invisible structures can be investigated.

Examination Of Neurologic Tissues

At present, detailed studies of the cytoarchitecture of the human brain are mainly possible through the laborious, irreversible methods of histology, which include numerous staining techniques for identifying different cell types and myelin content. However, these histological results do not translate well to in vivo MR imaging studies due to differing length scales and mechanisms of contrast. High-field MR imaging of ex vivo samples can help bridge the gap between these two disparate fields. At high resolution, MR, with its numerous forms of intrinsic and potential extrinsic contrast, is a promising tool to assess the myeloarchitecture within cortical layers as well as the cytoarchitecture of neuronal populations comprising functional areas. Recent advances in imaging technology permit isotropic resolutions of ex vivo samples below 100 microns. Despite high SNR in these images, often there is not sufficient inherent contrast to resolve laminar structure or neuronal subpopulations.

Just as conventional histologists rely on an arsenal of stains to identify cells under a microscope, cell- and tissue-specific magneto-optical contrast agents are described herein that can selectively improve the contrast of regions of interest. These novel contrast agents will permit MR to identify Brodmann and/or pathological areas pertinent to neurological diseases. Use of specialized paramagnetic cores will enhance tissue contrast in accordance with regional changes in relaxation parameters, e.g., allowing differentiation between gray and white matter, and various neural substructures.

The invention is further described in the following examples, which do not limit the scope of the invention described in the claims.

### EXAMPLES

### Example 1

### Standard Contrast Agents Do Not Selectively Reduce Relaxation Times of Specific Tissue Classes

An ideal ex vivo imaging MR contrast agent would selectively reduce the T1 or T2 of a specific tissue class or have a much greater effect on one tissue class than others. Relaxation parameters were analyzed for diethylenetriaminepentaacetate (DTPA⁵⁻) chelate of gadolinium(III) (Gd-DTPA), an agent that has been reported to show improvement in signal-to-noise ratio.

1) Gadolinium relaxation measurements ex vivo at 7T

Multi-echo fast low-angle shot (FLASH) acquisitions were performed on ex vivo tissue samples of formalin-fixed parietal cortex, immersed in 0 mM, 1 mM, 5 mM, or 10 mM concentrations of Gd-DTPA (Magnevist, Berlex Laboratories) diluted with phosphate-buffered saline (PBS). Samples soaked for one week and were then blotted and immersed in perfluoropolyether (Fomblin, Ausimont USA, Thorofare, N.J.) in a low-pressure environment. The perfluoropolyether is used as an embedding material because it does not contain hydrogen atoms that would contribute to the MR signal. The low-pressure environment is used to remove artifact-inducing air bubbles from within the tissue.

Three data sets were acquired in a three-turn, 3 cm solenoid coil in a 7T 90-cm bore human scanner (Siemens Medical Solutions, Erlangen, Germany) using flip angles of 10, 20, and 30 degrees, a TR of 76 ms and four echoes with a TE of 4.6, 8.6, 12.6, or 16.6 ms. Relaxation times T1 and T2* as well as proton density (Mo) were then estimated using the following equation:

$${SI}_{FLASH} = \frac{{M}_{o} sin ϑ \cdot ( 1 - {e}^{- TR / {T}_{1}} )}{1 - cos ϑ \cdot {e}^{- TR / {T}_{1}}} {e}^{- TE / {T}_{2}^{*}}$$

2). Gadolinium relaxation measurements ex vivo at 14T

Additional scans of ex vivo formalin-fixed parietal cortex were acquired using a 10-mm birdcage coil in a vertical 89-mm bore 14T magnet (Magnex Scientific, Oxford, England) with a 100 G/cm gradient coil. Samples were immersed in 0 mM, 0.25 mM, 0.5 mM, or 0.75 mM concentrations of Gd-DTPA diluted with PBS. Samples soaked between 24 hours and three months and were then blotted and immersed in perfluoropolyether in a low-pressure environment. T2* was too short to employ the same FLASH methods used at 7T. Thus, an inversion-recovery prepared spin-echo sequence and a multi-echo spin-echo sequence were used to determine T1 and T2, respectively. Parameters were estimated in gray and white matter using a nonlinear least-squares fitting routine with the following equations, where a represents flip angle deviation from 180o:

SI_{IR-SE}=Mₒ(1−2αe^{-TIIT}^{₁})SI_{SE}∝Mₒe^{-TEIT}^{₂}

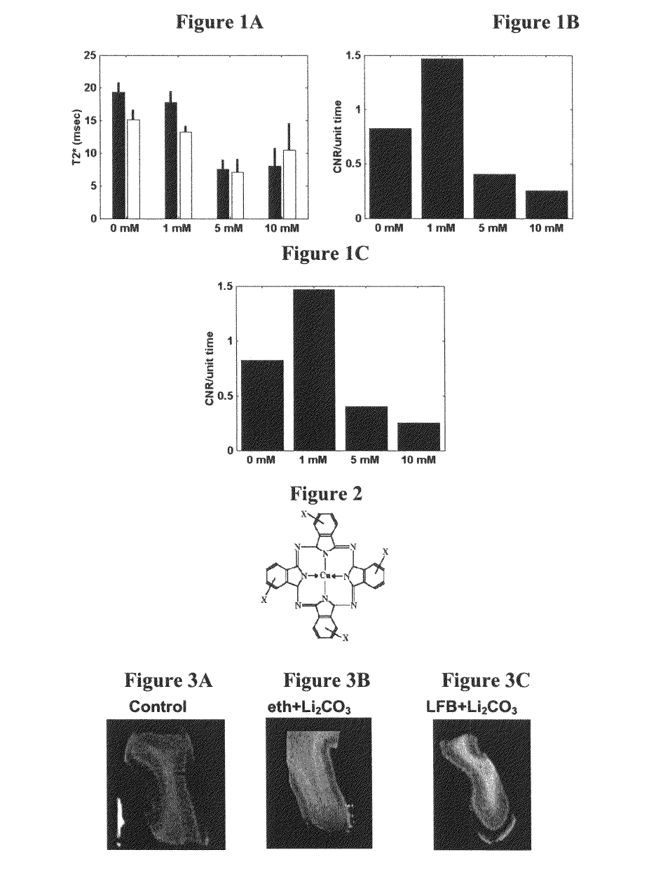

As FIGS. 1A-C show, Gd-DTPA shows a similar relative change in the relaxation parameters of gray and white matter, and therefore is not tissue specific in the brain.

### Example 2

### Luxol Fast Blue Acts as A Myelin-Specific MR Contrast Agent

The relaxation enhancement of white matter via the myelin stain luxol fast blue (LFB) was evaluated.

LFB is a tissue-specific histological dye that contains the arylguanidinium salts of anionic chromogens (Kiernan, Histological & Histochemical Methods: Theory and Practice. Oxford: Pergamon Press, 1990); its structure is shown in FIG. 2. LFB stains the phospholipids of fixed tissue samples (Salthouse, Nature, 199:821 (1963)) as well as the hydrophobic domains of protein molecules (Clasen et al., J. Neuropathol. Exp. Neurol., 32:271-283 (1973). The arylguanidinium cation is liberated in the solvent while attaching to the substrate, leaving the colored anion behind (Kieman, 1990). The staining is then differentiated and made specific for myelin by treatment with dilute aqueous lithium carbonate via an unknown reaction (Kiernan, 1990). This histological procedure is typically used for tissue slices 20-100 μm in thickness.

Formalin-fixed samples of visual cortex, approximately 1 cm in thickness, were immersed in a mixture of LFB dissolved in a solvent of 95% ethanol (500 mg LFB in 50 ml ethanol) for durations between 48 hours and 3 months. Following LFB staining, the tissue was rinsed in 70% ethanol, rinsed in distilled water, and then immersed in an aqueous solution of lithium carbonate(Li2CO3) (0.25 g Li2CO3 in 500 mL water) until gray-white matter contrast had increased, ranging between approximately 5 minutes and 24 hours. Next, the tissue was rinsed twice more in 70% ethanol and lastly in distilled water. Additional control samples were prepared with 1) formalin only or 2) 95% ethanol followed by immersion in Li2CO3.

Tissue samples were blotted and then immersed in perfluoropolyether (Fomblin, Ausimont USA, Thorofare, N.J.) in a low-pressure environment. The perfluoropolyether is used as an embedding material because it does not contain hydrogen atoms that would contribute to the MR signal. The low-pressure environment is used to remove artifact-inducing air bubbles from within the tissue.

Data was acquired using a 10 mm birdcage coil in a vertical bore 14T magnet (Magnex Scientific, Oxford, England), with an 89-mm bore and 100 G/cm gradient coil. An inversion-recovery prepared spin-echo sequence and a multi-echo spin-echo sequence were used to determine T1 and T2, respectively. Parameters were estimated in three regions of interest (ROIs), outer gray matter (GMo), inner gray matter (GMi), and white matter (WM), using a nonlinear least-squares fitting routine using the following equations, a:

SI_{IR-SE}=Mₒ(1−2αe^{-TIIT}^{₁})SI_{SE}∝Mₒe^{-TEIT}^{₂}

where α represents flip angle deviation from 180° and Mo is the equilibrium magnetization.

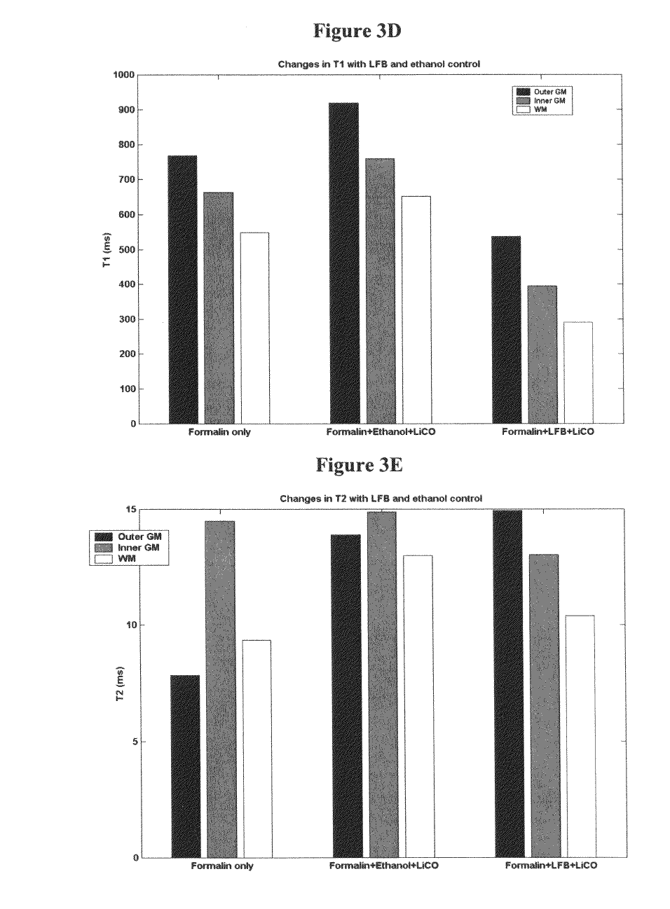

The results, shown in FIGS. 3A-C, indicated that the dye had penetrated deeply enough to change the relaxation times of the interior white matter in a 1 cm-thick slab of visual cortex. FIGS. 3A-C display inversion-recovery prepared spin echo images of samples that have been immersed in fixative only, fixative followed by 70% ethanol and lithium carbonate, and LFB plus lithium carbonate, respectively. The contrast in both sets of images, and thus the relaxation times, of tissues treated with LFB plus lithium carbonate differ from the control tissue.

As shown in FIGS. 3D-E, the ethanol-Li2CO3 mixture was found to increase T1 in GMo by 20%, in GMi by 14% and in WM by 19%, whereas the LFB mixture decreased T1 in GMo by 30%, in GMi by 41% and in WM by 47%. The T1 of WM underwent the largest change.

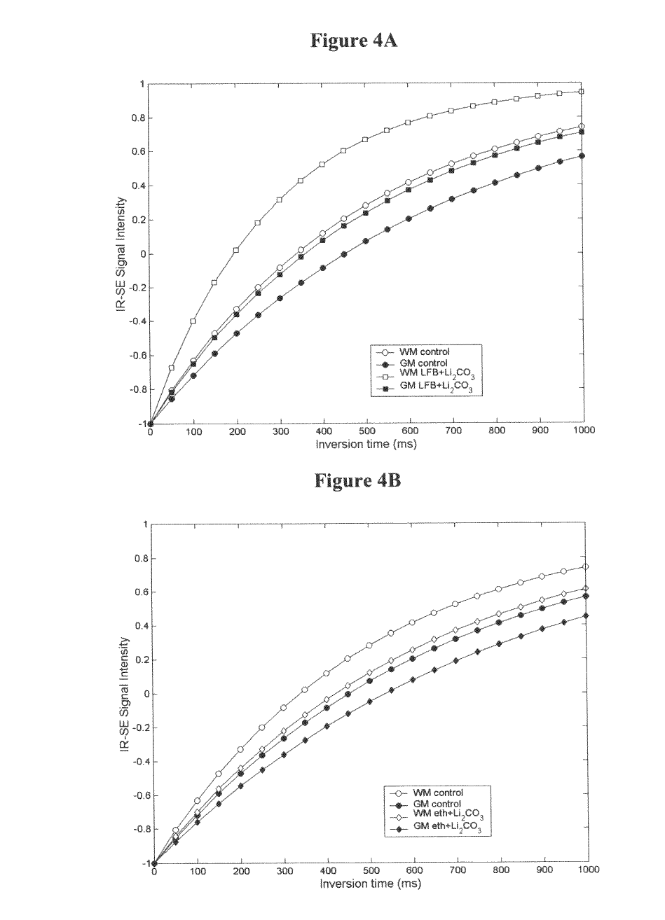

Longitudinal relaxation values were fit by non-linear least squares and are displayed in Table 1. These values were used to generate the longitudinal recovery curves shown in FIGS. 4A-B. Treatment with LFB and lithium carbonate was found to separate the T1 values in gray and white matter more than formalin fixation alone or treatment with only ethanol and lithium carbonate.

TABLE 1
Longitudinal relaxation values of white and gray matter

| Tissue Type and Preparation | T1 (ms) |
|---|---|
| WM control | 489.7 |
| GM control | 654.6 |
| WM eth + Li₂CO₃ | 610 |
| GM eth + Li₂CO₃ | 778 |
| WM LFB + Li₂CO₃ | 297.7 |
| GM LFB + Li₂CO₃ | 519.5 |

### Example 3

### Histological Validation of Specificity of LFB for White Matter

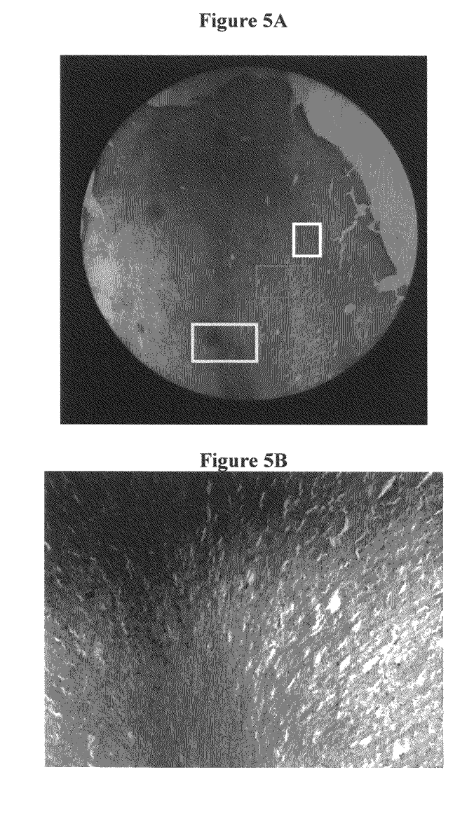

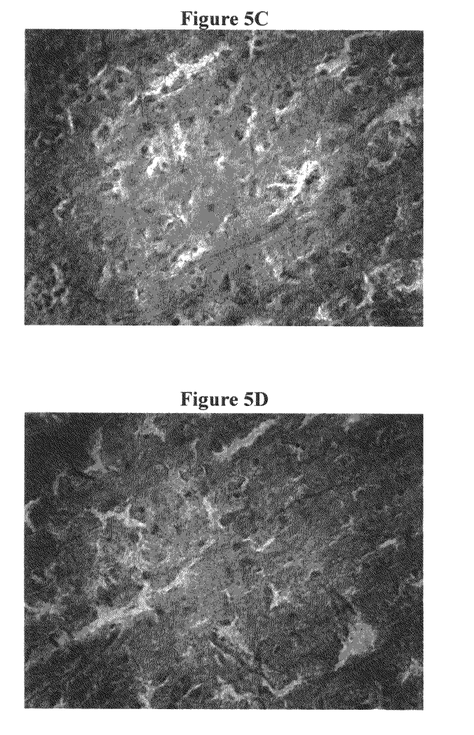

Samples of formalin-fixed, human visual cortex that had previously been immersion-stained with LFB and MR imaged were sectioned into 40-micron slices using a freezing microtome. Slices were mounted, coverslipped, and viewed under a microscope at 2×, 10×, and 40× magnification. The results are shown in FIGS. 5A-5D. The dark blue net of fibers, shown in FIG. 5B, and the individual fibers shown in FIGS. 5C and 5D illustrate the myelin-specific binding of LFB.

### Example 4

### Comparison of CNR for Various Contrast Agents

The contrast-enhancing properties of LFB were compared to that of standard imaging contrast agents, Gd-DTPA and manganese chloride (MnCl2) and formalin fixation alone (no additional contrast agent). The concentrations of Gd-DTPA and MnCl2 that maximize the gray-white matter contrast-to-noise ratio (CNR) at 14T, and estimated relaxation times for one gray matter compartment and one white matter compartment, were determined. The efficacy of LFB was compared to other agents by generating longitudinal recovery curves using the T1s and T2*s previously measured. A mean T1 value was calculated for the two discernable regions of gray matter in the LFB image.

The estimated parameters were then used with simulations of the steady-state Bloch signal equation to determine the TR, TE, and flip angle that maximize the CNR per unit time between any two regions of interest (ROIs). TR was varied between 40 and 100 ms, TE between 4 and TR-2 ms, and flip angle between 1 and 90 degrees. For each TR and flip angle combination, the CNR was computed by calculating the absolute value of the difference between ROIs in the synthesized signal. The CNR was assumed to decrease with square root of TR, corresponding to the assumption that the noise is time-independent (that is, a longer TR implies that fewer scans can be collected and averaged to increase SNR). Signal intensities were calculated using the following equation for a fast low-angle shot (FLASH) sequence:

$${SI}_{FLASH} = \frac{{M}_{o} sin ϑ \cdot ( 1 - {e}^{- TR / {T}_{1}} )}{1 - cos ϑ \cdot {e}^{- TR / {T}_{1}}} {e}^{- TE / {T}_{2}}$$

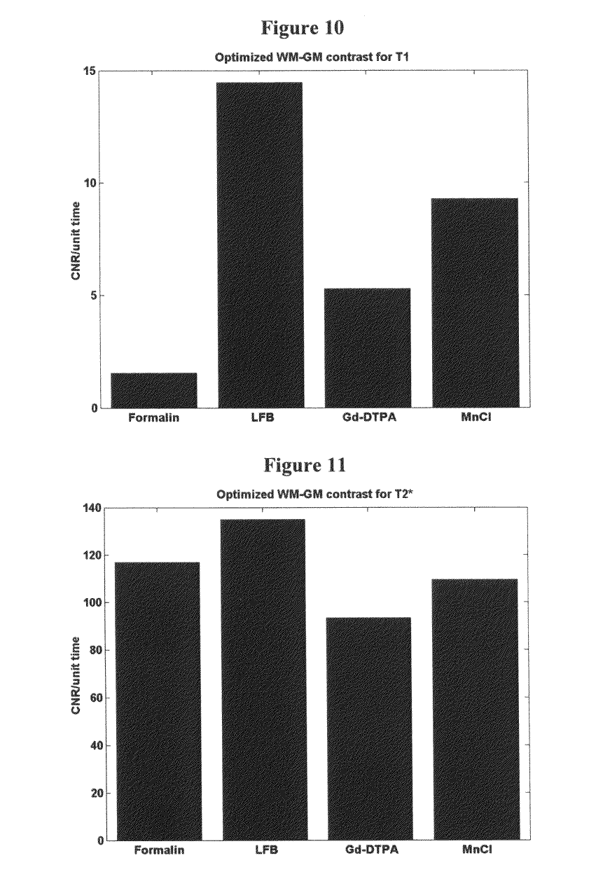

The results are shown in FIGS. 10 and 11. LFB-treated samples yielded the larger CNR then Gd- or MnCl-prepared samples in T1- and T2*-weighted simulations. The gain in CNR for LFB was much larger for T1-weighting.

### Example 5

### Gadolinium-Thionine is a Novel Neurospecific MR Contrast Agent

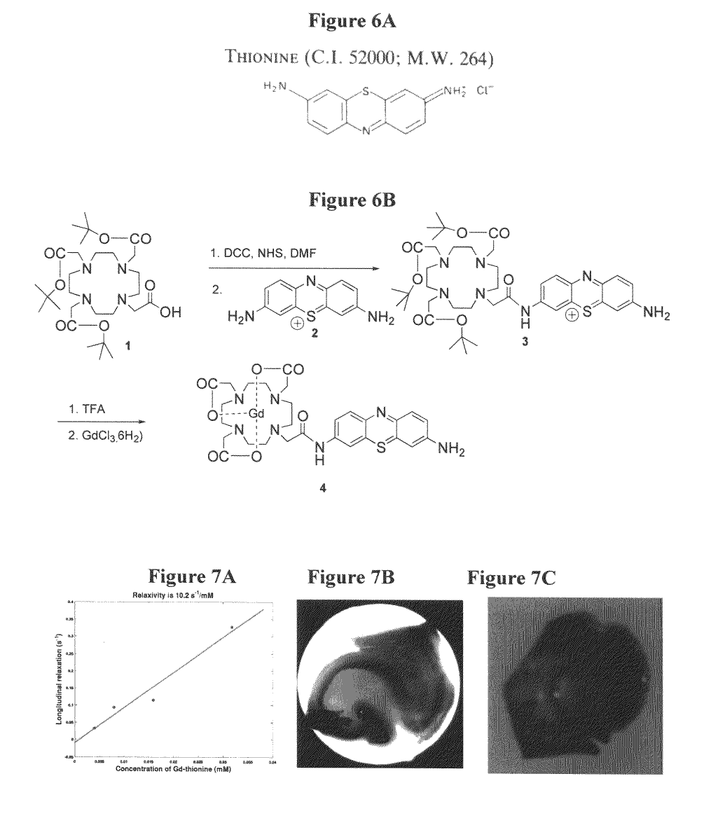

Thionine is a thiazine dye used for Nissl stains, which highlight neuronal cell bodies and beginnings of dendrites. The dye binds to the endoplasmic reticulum of cells, namely the acid groups in ribonucleic acid (Kiernan, 1990). The structure of thionine is shown in FIG. 6A.

To make thionine MR-visible, the dye was first attached to a chelate and then gadolinium chloride was added. The schematic for the procedure is shown in FIG. 6B. Specifically, 4,7,10-Tris-tert-butoxycarbonylmethyl-1,4,7,10-tetraaza-cyclododec-1-yl)-acetic acid (DOTA) was treated with excess dicyclohexylcarbodiimide (DCC) and N-hydroxysuccinimide (NHS) in dimethylformamide (DMF) in the presence of diisopropylethyl amine (DIEA) followed by the addition of thionine. The reaction mixture was stirred at room temperature for one day, filtered, and DMF was removed to complete dryness. The residue was treated with gadolinium chloride hexahydrate after Boc group deprotection in TFA for one day to afford the product. Mass spectroscopy was performed (MALDI-TOF MS) (M+H)⁺; the chemical formula, C₂₈H₃₃GdN₇O₇S, predicts a molecular weight of 769.92 and 769.17 was measured.

Four concentrations of gadolinium-thionine (GdT) were used to measure the agent's longitudinal relaxivity at a field strength of 14 Tesla. Inversion-recovery prepared spin-echo sequences were used to determine a value of 10.2 s⁻¹/mM in solution; data is shown in FIG. 7A. The specificity of the compound was tested in a 400-micron thick section of rat hippocampus. The appearance of the GdT-stained tissue in FIG. 7B (microscope image, 2× magnification) appears very similar to tissue stained with thionine in FIG. 7C (digital camera image). The darkest region in both preparations is the dentate gyrus, which contains the densest region of neurons. Also, the innermost region of white matter remains unstained.

### Example 6

### Variant Gadolinium-Thionine MR Contrast Agents

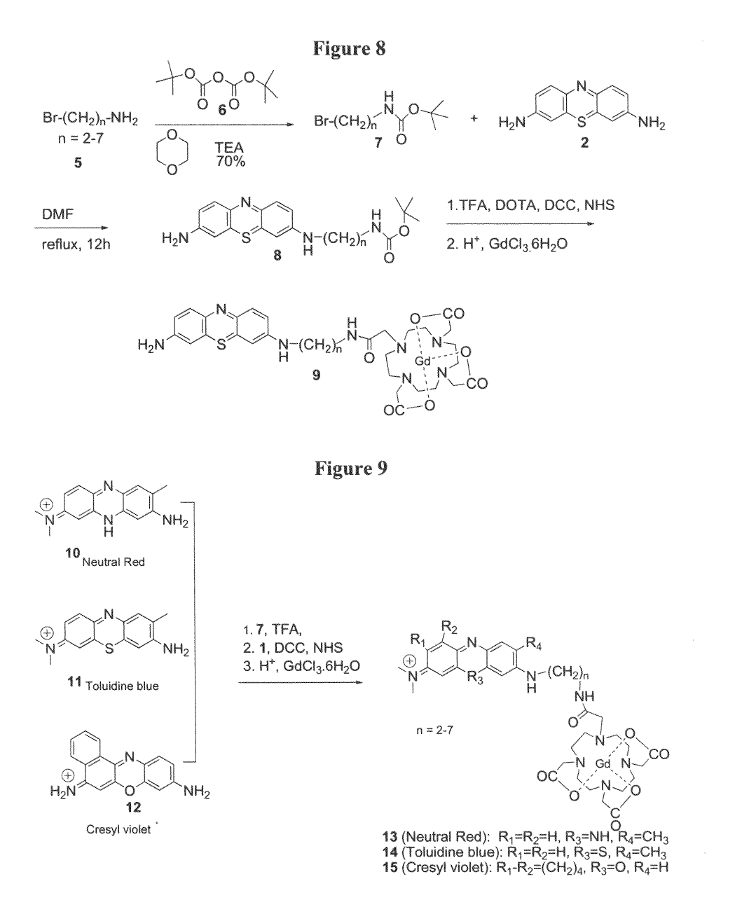

As shown in FIG. 8, labeling of DOTA to thionine can also be achieved using linkers, e.g., compounds 9 where the linker is from 2 to 7 carbon chain length. The synthesis starts with protection of the commercially available bromoalkylamine 5 with a Boc group using Di-ter-butyl dicarbonate 6 in dioxane in the presence of triethylamine. Alkylation of thionine 2 is be carried out under reflux for 12 hours to afford compound 8. The Boc group is deprotected in TFA, further coupling to the previously activated DOTA for one day. Then, all the Boc group on DOTA is deprotected before association with Gadolinium to afford the final product 9.

### Example 7

### Library of MR Contrast Agents

A library of probes that contain the optical dyes known for neuron staining can be synthesized using the same or similar methods as described herein, e.g., in Examples 3 and 4. For example, as shown in FIG. 9, the dyes neutral red 10, toluidine blue 11, and cresyl violet 12 are linked to a DOTA-Gd complex via a space linker 7, e.g., with a carbon chain length that varies from 2 to 7.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for obtaining an ex vivo magnetic resonance image of a specific tissue, the method comprising:
contacting a sample comprising the tissue with a contrast agent comprising a paramagnetic core and a tissue-specific histological dye, and
imaging the sample using magnetic resonance, thereby obtaining an image of the tissue.

2. The method of claim 1, wherein the contrast agent comprises luxol fast blue.

3. The method of claim 1, wherein the contrast agent comprises a paramagnetic core chemically linked via a chelating moiety to a tissue-specific histological dye.

4. The method of claim 3, wherein the contrast agent comprises gadolinium and thionine.

5. The method of claim 1, wherein the contrast agent selectively reduces relaxation parameters of the sample when compared to a control sample.

6. The method of claim 5, wherein the relaxation parameter reduced is spin-lattice relaxation time (T1), or spin-spin relaxation time (T2).

7. The method of claim 1, wherein the contrast agent selectively increases signal-to-noise ratio of the imaged sample when compared to a control sample imaged in the absence of the contrast agent.

8. The method of claim 1, further comprising performing a histological examination of the sample.

9. The method of claim 1, wherein the tissue-specific histological dye is selected from the group consisting of cresyl violet, toluidine blue, neutral red, thionine, and chromoxane cyanine R.

10. The method of claim 1, wherein the paramagnetic core is a rare earth metal.

11. The method of claim 1, wherein the paramagnetic core is a lanthanide.

12. The method of claim 1, wherein the paramagnetic core is selected from the group consisting of copper, gadolinium, manganese, and iron.

13. The method of claim 1, wherein the paramagnetic core is chemically linked via a chelating moiety to the tissue-specific histological dye, and the chelating moiety is a linear or macrocyclic chelating moiety.

14. The method of claim 1, wherein the paramagnetic core is chemically linked via a chelating moiety to the tissue-specific histological dye, and the chelating moiety is selected from the group consisting of 1,4,7,10-tetraazacyclododecane-N,N′,N″,N′″-tetraacetic acid (DOTA), ethylenediamine tetraacetic acid (EDTA), and diethylene triamine pentaacetate (DTPA).

15. The method of claim 1, wherein the tissue-specific histological dye is a neural tissue-specific dye.

16. The method of claim 15, wherein the neural tissue-specific dye preferentially stains one or more of white matter, gray matter, neurons, neuronal cell bodies, axons, glia, or myelin.

17. The method of claim 1, wherein the contrast agent includes thionine.

18. The method of claim 1, wherein the contrast agent includes C₂₈H₃₃GdN₇O₇S.

19. A method of making a contrast agent, the method comprising chemically linking a paramagnetic core to a tissue-specific histological dye via a chelating moiety, thereby forming the contrast agent.

20. The method of claim 19, comprising linking the chelating moiety to the tissue-specific histological dye via a linker comprising one or more carbon atoms.
